(19) United States
(12) Patent Application Publication
(10) Pub. No.: US 2013/0204232 A1
(43) Pub. Date: Aug. 8, 2013

(54) Unknown

(71) Applicants: Juergen Wieser, Ober-Ramstadt (DE); Roy Waeber, Langen (DE)

(72) Inventors: Juergen Wieser, Ober-Ramstadt (DE); Roy Waeber, Langen (DE)

(21) Appl. No.: 13/739,230

(22) Filed: Jan. 11, 2013

(30) Foreign Application Priority Data
Jan. 13, 2012 (DE) 10 2012 100 292.4

(51) Int. Cl. A61M 25/01

(52) U.S. Cl. CPC A61M 25/0108
USPC 604/528

(57) ABSTRACT

The invention relates to a medical device, comprising a hose-, tube- or hollow needle-shaped body, comprising a first layer (12) and an adjoining second layer (14) made of plastic material, wherein hollow spheres (22, 24) for generating echogenic properties are contained in the second layer. So as to achieve good echogenic properties, according to the invention both the inner first layer (12) and the adjoining second layer (14) comprise hollow spheres (18, 20, 22, 24) for generating echogenic properties, the hollow spheres (18, 20) of the first layer, these being the first hollow spheres, on average have smaller diameters D₁ than the hollow spheres (22, 24) of the second layer referred to as the second hollow spheres, and/or that the fill content F₁ of the first hollow spheres in the first layer is less than the fill content F₂ of the second hollow spheres in the second layer.

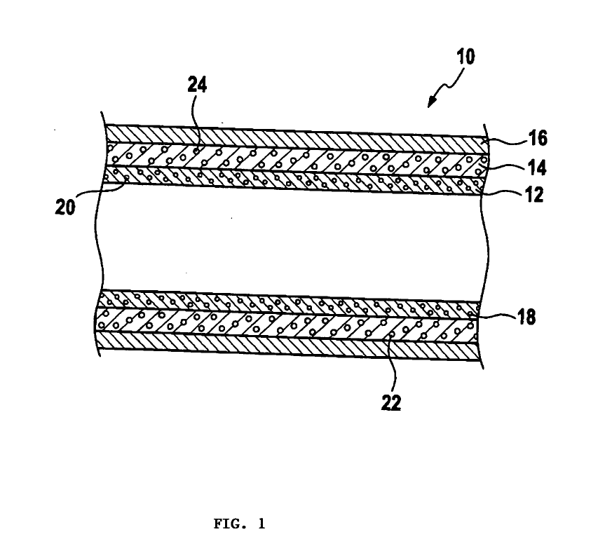

The invention relates to a medical device, comprising a hose-, tube- or hollow needle-shaped body, or having a shape that corresponds to such a body, comprising at least one inner first layer made of plastic material and an adjoining second layer made of plastic material, wherein hollow spheres for generating echogenic properties are contained in the second layer.

Over the last few years, sonography has strongly developed as an imaging method. As part of monitoring invasive procedures in the medical field, it provides a tracking option of the process that is not harmful to the patient, while also giving the physician the option to intervene. However, the catheters that are presently available on the market can be visualized by way of ultrasound only at depths of a few millimeters beneath the skin surface.

Thermoplastically processable plastic materials such as TPE, polyamide, polyurethane, polyethylene or soft PVC are employed in the production of medical catheter tubes. Multi-layer or composite tubes are also employed as catheter tubes so as to combine different materials in one tube. A special form of the multi-layer or composite tube is the liner tubes. These tubes comprise one or more integrated strips. These strips can be only color strips or functional strips, for example radiographic contrast strips.

In addition, multi-lumen tubes are known, which comprise several parallel tubes.

These have another field of application in medical technology, be it as infusion catheters, as feed tubes or as multi-functional tubes.

Moreover, hoses reinforced with woven fabric or hoses comprising wires/data lines integrally extruded therein are known. So as to contact sensors or electrodes, for example, electrical wires are known to be integrally extruded into the walls of a catheter tube.

When diagnostic and treatment procedures are carried out, accompanying imaging methods are employed for monitoring purposes so as to be able to determine or correct the location of the catheter during the procedure. X-rays are frequently used for this purpose so as to determine the locations of the catheters. Because pure plastic catheters are difficult to localize using X-rays, or cannot be localized at all, the previously described liner tubes are employed. In addition to the radiation, however, radiographic methods have the drawback that the catheter cannot be located without difficulty during the procedure, because these are temporally and locally decoupled processes.

In contrast, sonography offers the possibility of tracking the process in a way that is not harmful to the patient, while also giving the physician the option to intervene. However, the catheters that are presently in use exhibit only low echogenicity, which is to say ultrasonic visibility, both in terms of the structure and the materials used.

Echogenicity is substantially dependent on the sound-reflecting property of the respective structure. Visibility using ultrasound is based on the difference of the product of density and sound velocity between the tissue on the one hand and the object to be detected, such as a catheter, on the other.

A variety of methods for increasing echogenicity in all metallic objects such as needles are known, which generally relate to the mechanical modification of the geometry of the needle or needle tip. Roughening, faceting the polished section or similar measures are known. In addition, the surfaces of metallic objects are coated. For this purpose, scattering and reflection centers are embedded.

An echogenic coating of a catheter is known from DE-U-20 2009 001 974. The coating is composed of a polymer comprising embedded hollow microspheres, which in turn can be filled with gas, such as isobutane. The hollow spheres can be composed of vinylidene chloride, for example. The coating can be applied by way of spraying.

A layer system comprising a polymer matrix is known from EP-B-0 941 128, wherein compressible trapped gas bubbles form during heating, which cause the ultrasound to be reflected. In addition, a contrast agent for X-ray images may be introduced.

An echogenic surface layer according to U.S. Pat. No. 5,383,466 contains gas bubbles in a polymer matrix.

A medical instrument according to U.S. Pat. No. 6,306,094 comprises a coating on the outside, in which discrete mobile bubbles are created during a reaction with a reactant.

A matrix comprising contrast-enhancing elements, which in turn change the reflection properties thereof depending on the temperature, is known from U.S. Pat. No. 6,749,554.

DE-A-100 50 199 describes a method for producing an areal implant comprising detectable elements. During the production of the elements, which can be detected by ultrasound, syntactic foam is extruded, in which preferably gas-filled hollow glass spheres are embedded.

DE-T-693 19 632 relates to a method, a device, and a material for amplifying ultrasound echos. Reflective particles are embedded in a base or matrix material, which surrounds an elongated tube. The reflective particles, and optionally a radiopaque material, are mixed into the matrix material before a catheter is shaped by way of extrusion.

According to EP-B-0 386 936, sound reflective particles are added to a matrix material before a catheter is produced by way of extrusion.

An echogenic coating comprising mobile gas bubbles for a catheter is known from DE-T-697 28 892.

EP-A-2 308 551 describes a needle for blocking nerves. The needle comprises a metal tube on the inside and is coated with a layer comprising echogenic particles on the outside.

A medical device, notably in form of a catheter, is known from EP-B-1 462 056, which comprises at least two layers produced by way of extrusion, of which the outer layer is thicker than the inner layer. Gas bubbles are dispersed within the outer layer. In contrast, the inner layer is substantially free from gas bubbles. The gas bubbles can be formed by expanding polymer microspheres.

US-A-2008/0154136 relates to a catheter having echogenic properties. The catheter comprises two layers that contain particles, the acoustic impedance of which is different from that of blood.

EP-A-2 308 551 describes a catheter comprising an echogenic layer.

The subject matter of EP-B-0 941 128 is an echogenic coating of a catheter, for example.

A catheter is known from U.S. Pat. No. 5,289,831. The catheter comprises a coating containing glass particles that have diameters between 1 and 50 μm.

US-A-2005/0074406 describes a catheter on which several layers are applied, wherein spheres, which can be filled with gas and are made of silicone, polymer or cellulose or another biocompatible material, are present in an inner layer and a layer extending thereon. These measures are intended to improve the echogenicity of the catheter.

If echogenic properties are achieved by way of coatings, in particular by means of gas bubbles, irregular reflection occurs as a result of the production process, because it is not possible to produce gas bubbles that have uniform dimensions so as to achieve optimal ultrasound reflection.

It is the object of the present invention to refine a device, in particular a catheter or catheter tube, of the type mentioned above so that the echogenic properties are improved over the prior art, so as to enable visualization even at greater depths.

In order to achieve the object, according to the invention essentially both the inner first layer and the adjoining second layer comprise hollow spheres for generating echogenic properties, the hollow spheres of the first layer, these being the first hollow spheres, on average have smaller diameters than the hollow spheres of the second layer, these being the second hollow spheres, and/or the fill content F₁ of the first hollow spheres in the first layer is less than the fill content F₂ of the second hollow spheres in the second layer.

The device in particular comprises three layers, of which at least the first layer and the adjoining second layer, this being the center layer, exhibit echogenic properties.

In the case of a three-layer device, the inner first layer and the outer layer, this being the third layer, should have a thickness D_{1,3}, where D_{1,3}≦80 μm, in particular 20 μm≦D_{1,3}≦60 μm, and preferably D_{1,3} being approximately 40 μm

In contrast, according to the invention the second layer adjoining the inner first layer preferably has a thickness D₂, where 50μ≦D₂≦150 μm. More particularly, the thickness D₂ of the second layer should be greater than both that of the first layer and that of the second layer.

Independently of the thicknesses of the individual layers, the overall thickness of the layers should not exceed a value of 200 μm to 300 μm.

The material of the individual layers should be identical. Thermoplastically processable materials that have biocompatible properties should be employed. Thermoplastic elastomers (TPE), polyamide, polyurethane, polyethylene or soft PVC shall be mentioned as being preferred.

The wall thicknesses of the hollow spheres should be less than 10 μm, wherein preferred values range between 0.5 μm and 8 μm, with a range between 1 μm and 3 μm being particularly preferred.

The fill content F₂ of the spheres of the second layer should be 2% by weight to 25% by weight, and preferably between 5% by weight and 15% by weight.

In contrast, the fill content F₁ of the first layer should be less than 12%, and more particularly range between 3% and 10%.

The sphere diameters of the first layer are on average preferably smaller than the sphere diameters of the adjoining second layer.

The hollow spheres are in particular hollow glass spheres, which are filled with a gas such as O₂ or SO₂. The hollow glass spheres should exhibit negative pressure, which should range between 0.5 bar and 0.9 bar, and more particularly be approximately 0.7 bar, in absolute terms.

The use of hollow glass spheres creates the advantage that these, compared to those made of a plastic material, maintain the shapes thereof during extrusion, and the variance, which is to say the distribution of the diameters, is within relatively narrow limits. These measures ensure that, on average, the spheres of the inner layer have smaller diameters than those of the adjoining second layer. However, this does not preclude some spheres that, on average, are smaller than the spheres of the first layer to be present in the second layer, and conversely some spheres that have diameters greater than some spheres of the second layer will be present in the first layer.

Independently thereof, according to the invention the device is produced in particular by co-extruding the layers. The device is preferably composed of three layers, of which the inner first layer and the adjoining center layer, this being the second layer, exhibit echogenic properties of the type described above.

According to a refinement of the invention, one of the layers, in particular the echogenic layer, additionally contains an X-ray contrast medium, such as barium sulfate powder. Contrast media in strip shape or sections of strips are also possible.

The device according to the invention thus not only offers particularly good echogenic properties, but also X-ray contrast.

Further details, advantages, and characteristics of the invention will not only be apparent from the claims and the characteristics disclosed therein—either alone and/or in combination with one another—but also from the following description of a preferred exemplary embodiment disclosed in the drawing.

The only FIGURE shows a longitudinal sectional view of a detail of a catheter tube 10 as an example of a medical device according to the invention, which—also by way of example—comprises three layers 12, 14, 16, which are preferably produced by way of co-extrusion. A thermoplastic elastomer is the material that is used for each layer, wherein in particular thermoplastic copolyamides (TPE-A) shall be mentioned.

The device may also contain only two layers, or four or more layers.

In keeping with the teaching according to the invention, hollow spheres in the form of hollow glass spheres 18, 20 and 22, 24 are embedded in the inner or first layer 12 and the adjoining center or second layer 14, respectively, whereby good echogenic properties are attained.

The hollow glass spheres 18, 20, 22, 24 have negative pressure and are filled in particular with the gas O₂ or SO₂. In absolute terms, the negative pressure should range between 0.5 bar and 0.9 bar, and more particularly it should be 0.7 bar.

Independently thereof, and in particular so as to optimize the echogenic properties, the diameters of the hollow glass spheres 18, 20 that are embedded in the first layer 12 are smaller than the diameters of the hollow glass spheres 22, 24 that are present in the adjoining second layer 14. The diameters of the spheres 18, 20 in the first layer 12, which are referred to as first hollow glass spheres, are in particular smaller than 25 μm, and more particularly between 5 μm and 20 μm, wherein the values are average values. The diameters of the second hollow glass spheres 22, 24, these being those spheres that are present in the adjoining second layer 14, should range between 10 μm and 80 μm, in particular between 20 μm and 50 μm, and preferably between 30 μm and 40 μm.

With respect to the fill content of the first and second hollow glass spheres 18, 20, 22, 24, a distinction is made between the layers such that the fill content F₁ in the first or inner layer 12 is less than 12%, and in particular between 3% and 10%, and the fill content F₂ in the adjoining second layer should range between 2% and 25%, and in particular between 5% and 15%.

The outer or third layer 16 and the inner or first layer 12 should have the same thickness D_{1,3}, which is in particular D_{1,3}<80 μm, and preferably ranges between 20 μm and 60 μm. Approximately 40 μm is the preferred value.

With regard to the center layer, this being the second layer 14, a value between 50 μm and 150 μm is the preferred thickness D₂, where D₂>D_{1,3}.

Independently thereof, the overall wall thickness of the layers 12, 14, 16, which is to say of the catheter 10 through which the lumen 26 extends, should range between 50 μm and 300 μm.

Moreover, a contrast medium can be introduced in one of the layers 12, 14 16. For example, the outer layer 16 may contain barium sulfate powder. Other contrast media are likewise possible, wherein these do not necessarily have to be present in the outer layer 16. The X-ray contrast medium can rather also be introduced in the center layer 14 or inner layer 12, or in any one of layers 12, 14, 16. Instead of using powder as the contrast medium, it is also possible to co-extrude larger particles such as strips.

Based on the teaching according to the invention, a mass-produced product, in particular in the form of a catheter or catheter tube, can be made available, which exhibits good echogenic properties, wherein reproducibility is assured by the co-extrusion process. Because of the good echogenic properties, use in sonography as the imaging method is possible, and use in areas that were previously closed to sonography is now also possible. Precise placement of the catheter or catheter tube is assured especially with local anesthesia or vessel puncture.

1. A medical device, comprising a hose-, tube- or hollow needle-shaped body, or having the shape thereof, comprising at least one first layer (12) made of plastic material and an adjoining second layer (14) made of plastic material, hollow spheres (22, 24) for generating echogenic properties being contained in the second layer,
characterized in that
both the first layer (12), this being the inner layer, and the adjoining second layer (14) comprise hollow spheres (18, 20, 22, 24) for generating echogenic properties, the hollow spheres (18, 20) of the first layer, these being the first hollow spheres, on average have smaller diameters D₁ than the hollow spheres (22, 24) of the second layer that form the second hollow spheres, and/or that the fill content F₁ of the first hollow spheres in the first layer is less than the fill content F₂ of the second hollow spheres in the second layer.

2. The medical device according to claim 1,
characterized in that
the device (10) comprises three layers (12, 14, 16), of which at least the first layer (12) and the adjoining second layer (14), this being the center layer, exhibit echogenic properties.

3. The medical device according to claim 1,
characterized in that
the hollow spheres (18, 20, 22, 24) are hollow glass spheres.

4. The medical device according to claim 1,
characterized in that
the first hollow spheres (18, 20) and/or the second hollow spheres (14, 16) are each filled with a gas having negative pressure, in absolute terms, between 0.5 bar and 0.9 bar, and in particular approximately 0.7 bar.

5. The medical device according to claim 4,
characterized in that:
the gas is O₂ or SO₂.

6. The medical device according to claim 1,
characterized in that
the first hollow spheres (18, 20) have average diameters D₁, where D₁≦25 μm, and more particularly 5 μm≦D₁≦20 μm.

7. The medical device according to claim 1,
characterized in that
the second hollow spheres (22, 24) have average diameters D₂, where 10 μm≦D₂≦80 μm, in particular 20 μm≦D₂≦50 μm, and preferably 30 μm≦D₂≦40 μm.

8. The medical device according to claim 1,
characterized in that
the first and/or second hollow spheres (18, 20, 22, 24), on average, have a wall thickness WD, where WD≦10 μm, in particular 5 μm≦WD≦8 μm, with 1 μm≦WD≦3 μm being particularly preferably.

9. The medical device according to claim 1,
characterized in that
the fill content F₁ of the first hollow spheres (18, 20) of the first layer (12) is F₁<12%, and more particularly 3%≦F₁≦10%.

10. The medical device according to claim 1,
characterized in that
the fill content F₂ of the second hollow spheres (22, 24) of the second layer (14) is 2% by weight≦F₂≦25% by weight, and more particularly 5% by weight≦F₂≦15% by weight.

11. The medical device according to claim 1,
characterized in that
the first layer (12) and/or, in the case of an at least three-layer device (10), the outer third layer (18) have a thickness D_{1,3}, where D_{1,3}≦80 μm, in particular 20 μm≦D_{1,3}≦60 μm, and preferably D_{1,3} approximately 40 μm.

12. The medical device according to claim 1,
characterized in that
the second layer (14) has a thickness D₂, where 50 μm≦D₂≦150 μm.

13. A medical device according to claim 11,
characterized in that
the thickness D₂ of the second layer (14) is greater than the thickness of both the first layer (12) and the third layer (16).

14. The medical device according to claim 1,
characterized in that
the layers (12, 14, 16) of the device (10) have an overall thickness DS, where 50 μm≦DS≦300 μm.

15. The medical device according to claim 1,
characterized in that
at least one of the layers (12, 14, 16) contains an X-ray contrast medium such as barium sulfate powder.

16. The medical device according to claim 1,
characterized in that
at least the echogenic layer (14), and preferably each layer (11, 14), is composed of the same base material, which is a thermoplastically processable plastic material, in particular a material from the group consisting of thermoplastic elastomers, thermoplastic copolyamides, polyamide, polyurethane, polyethylene and soft PVC.

17. The medical device according to claim 1,
characterized in that
the device (10) is produced by co-extruding the layers (12, 14, 16).

18. A medical device according to claim 12,
characterized in that
the thickness D₂ of the second layer (14) is greater than the thickness of both the first layer (12) and the third layer (16).
